(19) United States
(12) Patent Application Publication
(10) Pub. No.: US 2011/0275873 A1
(43) Pub. Date: Nov. 10, 2011

(54) METHOD FOR PRODUCING AROMATIC HYDROCARBON

(75) Inventors: Yo Yamamoto, Shizuoka (JP); Tomohiro Yamada, Shizuoka (JP)

(73) Assignee: MEIDENSHA CORPORATION

(21) Appl. No.: 13/144,803

(22) PCT Filed: Jan. 15, 2010
(86) PCT No.: PCT/JP2010/050379

§ 371 Date: Jul. 15, 2011

(30) Foreign Application Priority Data
Feb. 12, 2009 (JP) 2009029704
Jul. 28, 2009 (JP) 2009174984

(51) Int. Cl. C07C 4/00

(52) U.S. Cl. 585/400

(57) ABSTRACT

[Task] To produce an aromatic hydrocarbon stably for a long time while maintaining a high aromatic hydrocarbon yield, when producing an aromatic hydrocarbon by a catalytic reaction of a lower hydrocarbon with a catalyst.

[Solving Means] An aromatic hydrocarbon is produced by providing a reaction step to obtain an aromatic hydrocarbon by conducting a catalytic reaction of a lower hydrocarbon with a catalyst and a regeneration step to regenerate the catalyst used in the reaction step, and by repeating the reaction step and the regeneration step. In the reaction step, carbon dioxide or carbon monoxide is added to the lower hydrocarbon, and the reaction temperature is made to be higher than 800° C.

### TECHNICAL FIELD

The present invention relates to a high-degree use of natural gas, biogas and methane hydrate, in which methane is a main component. In particular, it relates to a catalytic chemical conversion technique for efficiently producing aromatic compounds, in which benzene and naphthalenes, which are raw materials of chemical products such as plastics, are main components, and a high-purity hydrogen gas, from methane.

### BACKGROUND TECHNIQUE

Natural gas, biogas, and methane hydrate are regarded as the most effective energy resources as global warming measures, and an interest in its use technique is increasing. Methane resource making use of its clean property attracts an attention as the next generation new organic resource and as a hydrogen resource for fuel cells.

As a process for producing hydrogen and aromatic compounds, such as benzene, from methane, one is known in which methane is reacted in the presence of a catalyst, such as Non-patent Publication 1. As the catalyst upon this, molybdenum supported on ZSM-5 is said to be effective.

However, even in the case of using these catalysts, there are problems that carbon is deposited in large amount and that conversion of methane is low. In particular, carbon deposition is a problem that is directly connected with deterioration phenomena of the catalyst.

In order to solve these problems, in Patent Publication 1, a mixed gas prepared by adding CO₂ or CO to methane is used in the catalytic reaction under a catalytic reaction temperature of 300° C. to 800° C. The addition of CO₂ or CO makes it possible to suppress deposition of carbon, prevent catalyst deterioration, and stably produce aromatics.

Furthermore, in Patent Publications 2 and 3, the aromatic production reaction and the reaction for regenerating a catalyst used in its production reaction are alternately switched to suppress the deterioration over time of the catalyst and to maintain the catalytic reaction. That is, a lower hydrocarbon as the reaction raw material and a hydrogen-containing gas (or hydrogen gas) for maintaining and regenerating the catalyst are switched periodically to be in contact with the catalyst.

### PRIOR ART PUBLICATIONS

### Patent Publications

Patent Publication 1: Japanese Patent Application Publication 11-060514
Patent Publication 2: Japanese Patent Application Publication 2003-026613
Patent Publication 3: Japanese Patent Application Publication 2008-266244

### Non-Patent Publications

Non-patent Publication 1: JOURNAL OF CATALYSIS, 1997, Volume 165, p. 150-161

### SUMMARY OF THE INVENTION

### Task to be Solved by the Invention

Of the task mentioned in the above conventional techniques, it is extremely important to solve the catalyst deterioration by carbon deposition, which is exemplified in Non-patent Publication 1, in order to produce aromatic hydrocarbons, etc. stably for a long time in a reaction system of particularly a fixed bed mode.

In Patent Publication 1, the catalyst life is greatly improved by adding CO₂ or CO, but the initial yield from the start of the reaction until obtaining the maximum benzene yield is greatly lowered. Therefore, it has been difficult to be applied to a process that is wished to achieve a high yield within a short period of time of 2 to 3 hours.

Furthermore, the methods described in Patent Publications 2 and 3 make it possible to use the catalyst for a long time of a unit of several days, since a regeneration is conducted before the catalyst deteriorates perfectly. In the methods described in Patent Publications 2 and 3, deterioration of the catalyst is striking, and the catalytic reaction and the regeneration reaction are repeated on a cycle of a relatively short period of time.

In Patent Publication 2, the catalytic reaction and the regeneration reaction are switched every 1 to 20 minutes. Furthermore, it is described in Patent Publication 3 that, when the reaction time is 5 minutes or longer, a difficulty removable coke is deposited, and that, since it is not possible to sufficiently regain the catalytic activity even by conducting a regeneration in case that a difficulty removable coke has been accumulated, the reaction time is made to be 4 minutes or shorter.

That is, when a methane conversion reaction is continuously conducted, in some cases, the deposited carbon is accumulated during the reaction, and its removal becomes impossible. The production mechanism of the deposited carbon is not yet perfectly clear, it is considered to be produced by a plurality of reaction mechanisms. Then, since it is difficult to remove this deposited carbon after the reaction for a long time, it is necessary to switch the catalytic reaction and the regeneration reaction on a cycle of a short period of time.

It becomes, however, a factor of lowering of energy efficiency to switch the catalytic reaction and the regeneration reaction on a cycle of a short period of time.

In the case of repeating the catalytic reaction and the regeneration reaction on a cycle of a short period of time, there occur time and thermal losses when switching the gas. If it is a reaction system having particularly a large-scale reaction tube, the influence is large.

Furthermore, since a methane aromatization reaction is an endothermic reaction, the catalyst temperature is lowered by the endothermic reaction at the initial stage of the reaction. Therefore, in case that the reaction is in a short period of time, it is necessary to conduct a heating for an increase until the reaction temperature in the regeneration step. Since the aromatization reaction is activated with a higher reaction temperature, the temperature decrease at the initial stage is abrupt, and it is susceptible to the influence of the catalyst temperature lowering by this endothermic reaction.

Since the maximum yield is greatly lowered at the initial reaction by the above-mentioned reason in the aromatic hydrocarbon production method described in Patent Publication 1, it is not suitable for practical use even if it is applied to the aromatic hydrocarbon production method described in Patent Publication 2. Therefore, in case that an aromatic compound, such as benzene, is industrially produced from methane by using a lower hydrocarbon aromatization catalyst, there is a strong demand for maintaining a high yield and making the reaction time as long as possible.

Accordingly, it is an object of the present invention to highly maintain the yield of aromatic hydrocarbon and make the catalytic reaction time as long as possible in a method for producing an aromatic hydrocarbon by a catalytic reaction of a lower hydrocarbon with catalyst.

### Means for Solving the Task

An aromatic hydrocarbon production method of the present invention for achieving the above object is characterized by that, in a method for producing an aromatic hydrocarbon by repeating a reaction step to obtain an aromatic hydrocarbon by conducting a catalytic reaction of a lower hydrocarbon with a catalyst and a regeneration step to regenerate the catalyst used in the reaction step, in the reaction step, carbon dioxide or carbon monoxide is added to the lower hydrocarbon, and the reaction temperature is made to be higher than 800° C.

As the catalyst, it is possible to cite a metallosilicate supporting thereon molybdenum, a metallosilicate supporting thereon molybdenum and zinc, and a metallosilicate supporting thereon molybdenum and magnesium.

In the reaction step, the reaction step may be switched to the regeneration step, based on variation of the catalyst temperature. Furthermore, in the reaction step, the reaction step may be switched to the regeneration step, based on yield of benzene produced in the reaction step.

Then, it suffices that the amount of addition of the carbon dioxide or carbon monoxide is 0.01% to 30% per volume of the lower hydrocarbon.

### ADVANTAGEOUS EFFECT OF THE INVENTION

According to the above invention, when producing an aromatic hydrocarbon by a catalytic reaction of a lower hydrocarbon with a catalyst, it is possible to produce an aromatic hydrocarbon stably for a long time while maintaining a high aromatic hydrocarbon yield.

### BRIEF DESCRIPTION OF THE DRAWINGS

FIG. 1 It is a graph showing benzene yield variations over time in case that catalytic reactions have been continuously conducted in the presence of a Zn/Mo-HZSM5 catalyst (with no addition of CO₂).

FIG. 2 It is a graph showing benzene yield variations over time in case that catalytic reactions have been continuously conducted in the presence of a Zn/Mo-HZSM5 catalyst (with the addition of CO₂ in 3%).

FIG. 3 It is a graph showing benzene yield variations over time in case that the catalytic reaction step and the regeneration step have been repeated.

FIG. 4 It is a graph showing benzene yield variations over time in case that an aromatic hydrocarbon and hydrogen have been produced from methane in the presence of Zn/Mo-HZSM5, Mo-HZSM5 and Mg/Mo-HZSM5 catalysts.

### EMBODIMENT FOR CONDUCTING THE INVENTION

The present invention is an invention related to a method for producing an aromatic hydrocarbon by reacting a lower hydrocarbon in the presence of a catalyst. It is characterized by that the reaction temperature is made to be higher than 800° C. and that the catalyst is regenerated by switching to a regeneration gas at regular intervals. In particular, it is characterized by that the maximum yield has improved dramatically by making the reaction temperature higher than 800° C.

In addition, while suppressing the occurrence of a striking carbon (coke) deposition by adding carbonic acid gas in an amount that does not become excessive at the time of the reaction (0.01-30%, preferably 0.1-6%), the catalytic reaction is conducted by switching to a regeneration gas at regular intervals. With this, the reaction is conducted for a long time with no accumulation of a difficultly removable coke, while maintaining a high yield.

The reactor used in the method for producing an aromatic hydrocarbon of the present invention is exemplified by a fixed bed reactor or flow bed reactor, etc.

In the present invention, as the metallosilicate having a catalyst metal supported thereon, for example, in the case of aluminosilicate, it is possible to cite molecular sieve 5A, faujasite (NaY and NaV, ZSM-5 and MCM-22, which are porous materials formed of silica and alumina. Furthermore, it can be exemplified by zeolite supports, which are porous materials having phosphoric acid as a main component and are characterized by having 6-13 angstrom micropores and channels, such as ALPO-5 and VPI-5. Furthermore, it can be exemplified by meso-porous supports, which contain silica as a main component and partly alumina as a component and are characterized by cylindrical micropores (channels) of meso-micropores (10-1000 angstroms), such as FSM-16 and MCM-41. Furthermore, besides the aluminosilicate, it is also possible to use a metallosilicate formed of silica and titania, etc. as the catalyst.

Furthermore, it is desirable that a metallosilicate used in the present invention has a surface area of 200-1000 m²/g and that its micro- and meso-pores are within a range of 5-100 angstroms. Furthermore, in case that the metallosilicate is, for example, aluminosilicate, it is possible to use one in which the ratio of silica content to alumina content (silica/alumina) is 1-8000, similar to porous materials that are generally available. It is, however, more preferable to make silica/alumina within a range of 10-100 in order to conduct an aromatization reaction of a lower hydrocarbon of the present invention with a practical lower hydrocarbon conversion and a selectivity to an aromatic compound.

Furthermore, in the case of supporting a catalyst metal (a precursor containing the same) of the present invention on a metallosilicate, it is conducted to have a weight ratio of the catalyst metal to the support of a range of 0.001-50%, preferably 0.01-40%. Furthermore, as a method of supporting on the metallosilicate, there is a method in which a supporting is conducted on a metallosilicate support by impregnation or an ion exchange method from a catalyst metal precursor aqueous solution or solution of an organic solvent such as alcohol, and then a heating treatment is conducted under an atmosphere of an inert gas or oxygen gas. As this method is explained in more specifically, firstly, for example, an impregnation supporting of an ammonium molybdate aqueous solution is conducted on a metallosilicate support, and the supported substance is dried to remove the solvent, and then a heating treatment is conducted at a temperature of 250-800° C. (preferably 350-600° C.) in a nitrogen-containing oxygen stream or a pure oxygen stream. With this, it is possible to produce a metallosilicate catalyst supporting molybdenum as a catalyst metal.

Then, it is preferable to use molybdenum as a catalyst metal of the present invention, but it is also possible to use rhenium, tungsten, iron, and cobalt. Of the catalyst metals, as examples of a molybdenum-containing precursor, it is possible to cite halides such as chlorides and bromides, mineral acid salts such as nitrates, sulfates and phosphates, carboxylates such as carbonates, acetates and oxalates, etc., besides ammonium paramolybdate, ammonium phosphomolybdate, 12-series molybdic acids.

As the metallosilicate, it is general to use a proton-exchanged type (H type). Furthermore, the proton may be partly exchanged for at least one cation selected from alkali metals such as Na, K and Li, alkali-earth elements such as Mg, Ca and Sr, and transition metal elements such as Fe, Co, Ni, Zn, Ru, Pd, Pt, Zr and Ti. Furthermore, the metallosilicate may contain a suitable amount of Ti, Zr, Hf, Cr, Mo, W, Th, Cu, Ag, etc.

The form of the metallosilicate catalyst supporting a catalyst metal is not particularly limited. It suffices to use one having an arbitrary shape such as powdery and granular. Furthermore, it is optional to use alumina, titania, silica, a clayey compound, etc. as the support or binder.

The metallosilicate catalyst supporting thereon a catalyst metal may be used by shaping into pellets or an extrusion after adding a binder such as silica, alumina and clay.

Furthermore, in the present invention, lower hydrocarbons mean methane and C₂₋₆ saturated and unsaturated hydrocarbons. These C₂₋₆ saturated and unsaturated hydrocarbons can be exemplified by ethane, ethylene, propane, propylene, n-butane, isobutane, n-butene, and isobutene, etc.

In the following, a further detailed explanation is conducted by examples.

### EXAMPLES

Using H-type ZSM-5 zeolite (SiO₂/Al₂O₃=40) as a metallosilicate support, a lower hydrocarbon aromatization catalyst (in the following, referred to as catalyst) was prepared by the following preparation method.

400 g of HZSM5 was added to an aqueous solution prepared by dissolving predetermined amounts of ammonium molybdate and zinc nitrate in 2000 ml of ion-exchanged water, followed by stirring at room temperature for 3 hours, thereby conducting an impregnation supporting of zinc and molybdenum on HZSM5. The obtained zinc/molybdenum-supported HZSM5 (Zn/Mo-HZSM5) was dried, followed by baking at 550° C. for 8 hours, thereby obtaining a catalyst powder. Furthermore, an inorganic binder was added to this catalyst powder, followed by extrusion into pellets and then baking to prepare a catalyst.

The catalyst prepared by the above method was put into a reaction tube (inner diameter 18 mm) made by an Inconel 800H, gas-contact portion calorizing treatment of a fixed bed flow-type reaction apparatus. The temperature of the inside of the reaction tube was made to be higher than 800° C., the pressure was set at 0.3 MPa, and a reaction gas containing methane was supplied at a flow rate of a space velocity of 3000 ml/g-MFI/h to examine catalytic activity of the lower hydrocarbon aromatization reaction using methane as the raw material. As to the evaluation of the catalyst, it was evaluated by yield of benzene relative to the lower hydrocarbon made to flow. Yield of benzene is defined as follows.

Benzene yield (%)={(the amount of benzene produced (mol))/(the amount of methane used for the methane reforming reaction (mol))}×100

In a pretreatment of the catalyst prior to supplying the reaction gas, the temperature of the catalyst was increased to 550° C. under air stream, followed by maintaining for 2 hours, then switching to a pretreatment gas of 20% methane:80% hydrogen, increasing the temperature to 700° C., and maintaining for 3 hours. After that, it was switched to the reaction gas, followed by increasing the temperature to a predetermined temperature (780° C., 800° C. or 820° C.) to conduct an evaluation of the catalyst.

In the regeneration step of the catalyst, the reaction temperature of the reaction tube was set the same as at the time of the reaction, the pressure was set at 0.3 MPa, and hydrogen gas was supplied at a flow rate of a space velocity of 3000 ml/g-MFI/h.

As to the analysis of hydrogen, argon and methane, the analysis was conducted by TCD-GC. As to the analysis of aromatic hydrocarbons such as benzene, toluene, xylene and naphthalene, the analysis was conducted by FID-GC.

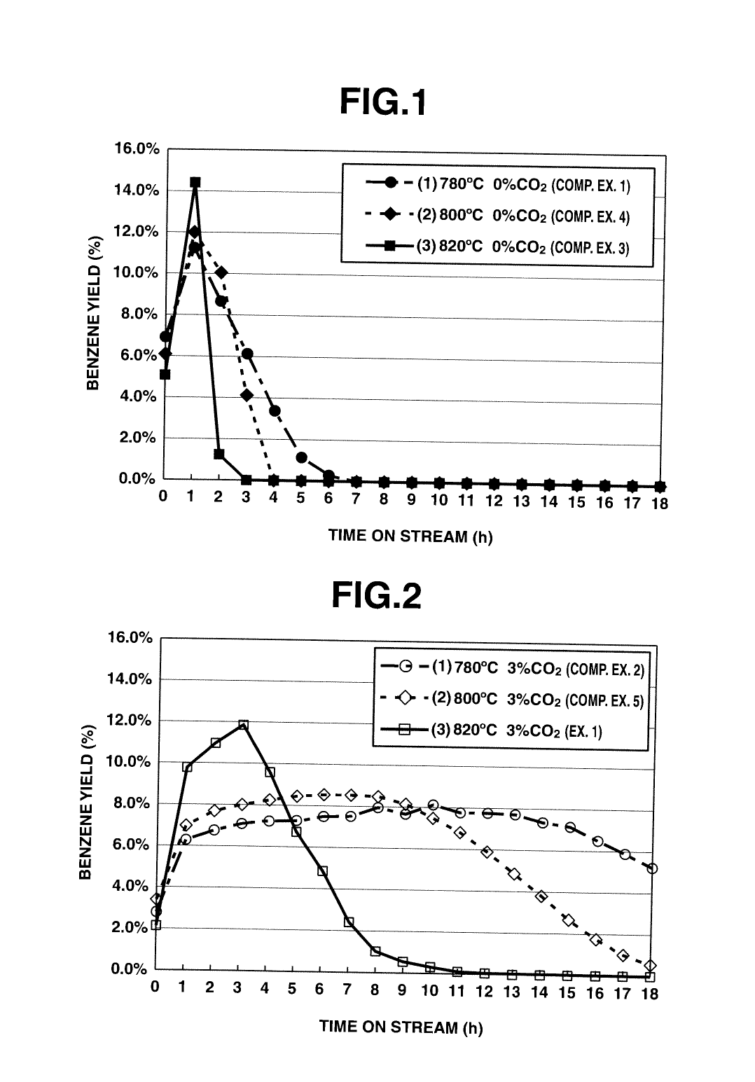

FIG. 1 is a graph showing benzene yield variations over time in case that catalytic reactions have been continuously conducted in the presence of a Zn/Mo-HZSM5 catalyst with no addition of CO₂ at respective temperature conditions of 780° C. (Comparative Example 1), 800° C. (Comparative Example 4) and 820° C. (Comparative Example 3). Furthermore, FIG. 2 is a graph showing benzene yield variations over time in case that catalytic reactions have been continuously conducted in the presence of a Zn/Mo-HZSM5 catalyst with the addition of CO₂ in 3% at respective temperature conditions of 780° C. (Comparative Example 2), 800° C. (Comparative Example 5) and 820° C. (Example 1).

In the following, the reaction gases and the reaction conditions of Comparative Examples 1-4 and Examples 1 and 2 are shown.

In Comparative Example 1, the reaction was conducted at a reaction temperature of 780° C. with no addition of carbon dioxide to 100 (volume) of methane as the reaction gas at the time of the reaction, and an observation over time of the analysis result was conducted.

In Comparative Example 2, the reaction was conducted at a reaction temperature of 780° C. with the addition of carbon dioxide by 3 (volume) to 100 (volume) of methane as the reaction gas at the time of the reaction, and an observation over time of the analysis result was conducted.

In Comparative Example 3, the reaction was conducted at a reaction temperature of 820° C. with no addition of carbon dioxide to 100 (volume) of methane as the reaction gas at the time of the reaction, and an observation over time of the analysis result was conducted.

In Comparative Example 4, the reaction was conducted at a reaction temperature of 800° C. with no addition of carbon dioxide to 100 (volume) of methane as the reaction gas at the time of the reaction, and an observation over time of the analysis result was conducted.

In Comparative Example 5, the reaction was conducted at a reaction temperature of 800° C. with the addition of carbon dioxide by 3 (volume) to 100 (volume) of methane as the reaction gas at the time of the reaction, and an observation over time of the analysis result was conducted.

In Example 1, the reaction was conducted at a reaction temperature of 820° C. with the addition of carbon dioxide by 3 (volume) to 100 (volume) of methane as the reaction gas at the time of the reaction, and an observation over time of the analysis result was conducted.

In comparison between Comparative Example 1 and Comparative Example 2, the catalytic activity is lost in 7 hours of the reaction time in case that the reaction was conducted with no addition of carbon dioxide (FIG. 1, Comparative Example 1), and in contrast the initial maximum benzene yield is maintained even in 15 hours of the reaction time by adding carbon dioxide (FIG. 2, Comparative Example 2).

However, the maximum benzene yield is 11% in case that carbon dioxide is not added (FIG. 1, Comparative Example 1), and in contrast the maximum benzene yield lowers greatly as it is 8% in case that carbon dioxide was added (FIG. 2, Comparative Example 2).

That is, in case that the reaction temperature is the same, the addition of carbon dioxide prolongs the time during which the catalyst maintains activity, but reduces the benzene production rate.

On the other hand, in comparison between Comparative Example 1 and Comparative Example 3, the maximum yield of benzene is 11% when the reaction temperature is 780° C. (FIG. 1, Comparative Example 1), and in contrast the maximum yield of benzene improves to 12% at 800° C. (FIG. 1, Comparative Example 4). Furthermore, the maximum yield of benzene improves dramatically as it becomes over 14% when the reaction temperature is set at 820° C. (FIG. 1, Comparative Example 3). However, the time during which the catalytic activity is maintained becomes short, and the catalytic activity is almost lost in 3 hours in Comparative Example 3.

That is, raising of the reaction temperature improves the maximum benzene yield, but also speeds up the rate at which the catalyst deteriorates.

Thus, like Example 1 shown in FIG. 2, when the catalytic reaction is conducted by adding CO₂ and setting the reaction temperature at 820° C., it showed the maximum benzene yield exceeding the maximum benzene yield at the time when the catalyst reaction was conducted under conditions of Comparative Example 1. That is, while maintaining a high activity, the catalyst stability also improved.

In FIG. 2, in comparison between Comparative Example 2 and Comparative Example 5, in Comparative Example 5, an improvement of benzene yield is found by setting the reaction temperature at 800° C., but lowering of stability of benzene yield is striking as compared with Comparative Example 2.

In Example 1, the catalyst stability lowers as compared with other Comparative Examples 2 and 5, but benzene yield improves dramatically. Therefore, it is suggested that the effect of a dramatic improvement of benzene yield can be obtained by conducting the reaction at a catalytic reaction temperature that is higher than 800° C.

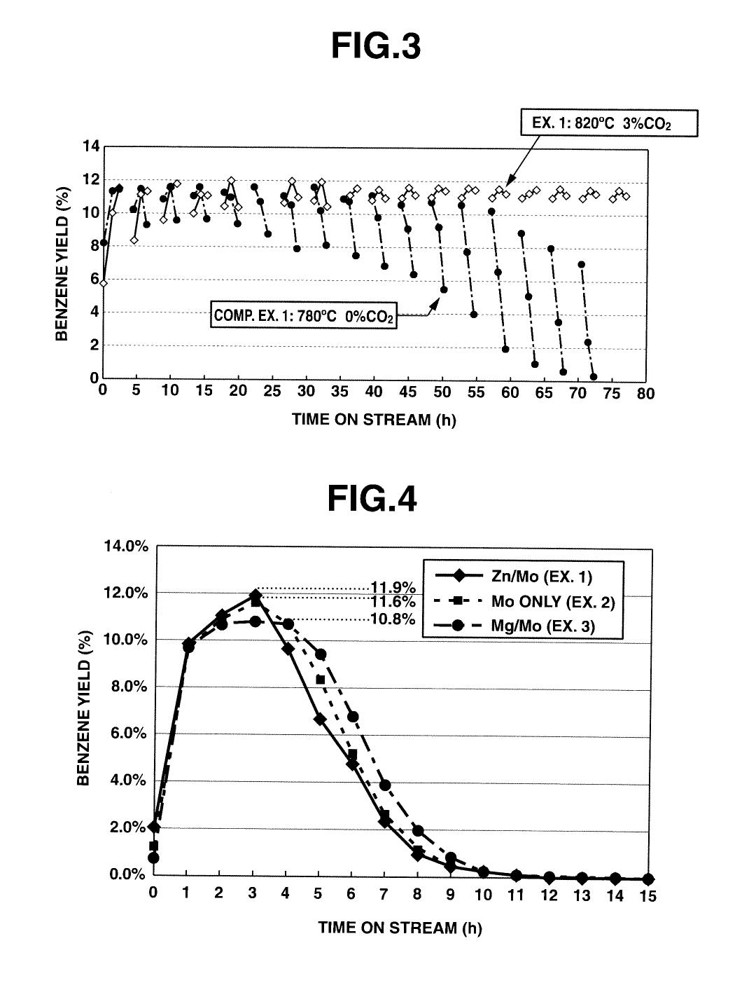

Next, FIG. 3 shows the results obtained by repeating a cycle of conducting the catalytic reaction (reaction step) for 2 hours under the reaction conditions of the reaction gas and the catalyst of Comparative Example 1 and Example 1 and then conducting the regeneration reaction (regeneration step) for 2 hours by hydrogen gas. Besides, the reactions in the regeneration step were conducted at the temperatures of the respective catalytic reaction steps.

As shown in FIG. 3, since benzene yield is greater than 10% after more than 80 hours (the catalyst working time: 40 hours) in the aromatic hydrocarbon production method by the conditions of Example 1, it is understood that an aromatic compound can be produced extremely stably with high yield.

On the other hand, in the case of repeating the reaction step to produce an aromatic hydrocarbon under conditions of Comparative Example 1 and the regeneration step to regenerate the catalyst used in the reaction, a tendency of deterioration is found in around 20 hours, and benzene yield lowers in 70 hours to about 60% of the maximum.

In comparison between benzene maximum yields of Comparative Example 1 of FIG. 1 and Example 1 of FIG. 2, both are around 12%. However, when the catalytic reaction step and the regeneration step are repeated, it is understood that catalyst stability is improved, while maintaining high benzene yield (catalytic activity), in the reaction conditions of Example 1, as compared with Comparative Example 1.

Furthermore, the catalytic reaction step and the regeneration step may be switched to each other, based on the temperature variation by measuring the temperature of the catalyst in the catalytic reaction step.

In the catalytic reaction step, since the aromatization reaction of the lower hydrocarbon is an endothermic reaction, the temperature of the catalyst lowers at the time of the reaction. Then, as the catalyst deteriorates, the aromatization reaction activity of the lower hydrocarbon also lowers. Therefore, it is possible to detect the degree of deterioration of the catalyst by measuring the temperature variation of the catalyst. Accordingly, it is possible to more efficiently produce an aromatic hydrocarbon and prevent deterioration of the catalyst by switching from the reaction step to the regeneration step after the temperature of the catalyst starts to increase.

Furthermore, it is also possible to save energy for increasing the catalyst temperature in the regeneration step to the preset temperature necessary for the reaction, by switching to the regeneration step once the temperature of the catalyst increases.

Furthermore, in the catalytic reaction step, the catalytic reaction step and the regeneration step may be switched, based on benzene yield. It is possible to prevent accumulation of a difficultly removable coke by switching from the catalytic reaction step to the regeneration step, prior to the time at which benzene yield changes from increase to decrease in the variation of benzene yield of FIG. 2.

Furthermore, there were examined catalytic activity differences due to differences of catalyst metals to be supported on HZSM5. The catalytic reactions were conducted under reaction conditions of a reaction temperature of 820° C., a pressure of 0.3 MPa, a methane reaction gas, space velocity of 3000 ml/g-MFI/h and the addition of CO₂ in 3% by using Mo-HZSM5 (Example 2) and Mg/Mo-HZSM5 (Example 3) as the catalysts.

As a method for producing Mo-HZSM5 catalyst, similar to Example 1, there was used a method in which 400 g of HZSM5 was added to an aqueous solution prepared by dissolving a predetermined amount of ammonium molybdate in 2000 ml of ion-exchanged water, followed by stirring at room temperature, thereby conducting an impregnation supporting of molybdenum on HZSM5.

Furthermore, as a method for producing Mg/MoHZSM5 catalyst too, similar to the catalyst production method used in Example 1, there was used a method in which HZSM5 was added to an aqueous solution containing molybdenum ions and magnesium ions to conduct an impregnation supporting of Mg and molybdenum on HZSM5.

The benzene yield variation over time with each catalyst is shown in FIG. 4. As shown in FIG. 4, it was possible to obtain a high benzene yield exceeding 10% by using any catalyst of Zn/Mo-HZSM5 (Example 1), Mo-HZSM5 (Example 2) and Mg/Mo-HZSM5 (Example 3).

The maximum benzene yield in the case of using Mo-HZSM5 (Example 2) as a catalyst is 11.6%. Although it is lower than that of the case of using Zn/Mo-HZSM5 as a catalyst, but it is superior in reaction stability.

On the other hand, the maximum benzene yield in the case of using Mg/Mo-HZSM5 (Example 3) as a catalyst is 10.8% and is the lowest as compared with the other examples, but is best in reaction stability. Reaction stability improvement is preferable, since it is possible to conduct a reaction of high benzene yield for a long time.

Furthermore, in the case of using any of Mo-HZSM5 and Mg/Mo-HZSM5 too, it was possible to continue the catalytic reaction under a high benzene yield condition for a long time similar to Zn/Mo-HZSM5 (FIG. 3, Example 1) by repeating the catalytic reaction step and the catalyst regeneration step.

In the case of using Mg/Mo-HZSM5 as a catalyst, however, it has been confirmed by experiments that lowering of benzene yield is found, as compared with the other catalysts (Examples 1 and 2), when the time of reacting by repeating the reaction step and the regeneration step exceeds 80 hours. That is, it is considered that a sufficient deposition of a difficultly removable coke cannot be prevented by Mg/Mo-HZSM5 in the regeneration step. Therefore, as shown in FIG. 4, even if they have the same degree of catalytic activity at the initial stage, we can say that Mo-HZSM5 and Zn/Mo-HZSM5 are more preferable catalysts in terms of being able to prevent deposition of a difficultly removable coke.

As mentioned above, it is possible to produce an aromatic hydrocarbon such as benzene with high yield by the method for producing an aromatic hydrocarbon and hydrogen using a lower hydrocarbon aromatization catalyst according to the present invention. That is, it is possible to suppress lowering of the maximum yield of benzene or the like, obtain a practically sufficient yield and maintain the catalytic activity for a long time by making the reaction temperature higher than 800° C. and adding CO₂ or CO.

That is, it is possible to dramatically improve benzene yield by making the reaction temperature higher than 800° C. and to suppress accumulation of a difficultly removable coke by adding CO₂. Since CO₂ has an effect of suppressing an aromatization reaction, it is possible to improve benzene yield (catalytic activity) by reducing the amount of addition of CO₂, but it becomes difficult to repeat the catalytic reaction and the catalyst regeneration reaction for a long time as in the present invention.

In particular, reaction yield at the initial stage becomes important in a process of repeating the catalytic reaction step and the catalyst regeneration step. Therefore, according to the aromatic hydrocarbon production method of the present invention, it is possible to obtain a high benzene yield, suppress the formation of a deposited carbon that is difficult of regenerative removal, and maintain a high catalytic activity for a long time even by repeating the catalytic reaction and the regeneration reaction.

Furthermore, the present invention is not limited to the examples, and it is optional to add carbon monoxide in place of carbon dioxide. Besides, it is possible to suitably select the reaction conditions such as flow rate of the reaction gas, the catalyst to be used (the type of catalyst to be supported and the amount of supporting), etc.

1.-7. (canceled)

8. A method for producing an aromatic hydrocarbon, comprising repeating (a) a reaction step to obtain an aromatic hydrocarbon by conducting a catalytic reaction of a lower hydrocarbon with a catalyst and (b) a regeneration step to regenerate the catalyst used in the reaction step,
wherein, in the reaction step, carbon dioxide or carbon monoxide is added to the lower hydrocarbon, and reaction temperature of the reaction step is made to be higher than 800° C.

9. The method according to claim 8, wherein the catalyst comprises a metallosilicate and a molybdenum supported on the metallosilicate.

10. The method according to claim 9, wherein the catalyst further comprises a zinc supported on the metallosilicate.

11. The method according to claim 9, wherein the catalyst further comprises a magnesium supported on the metallosilicate.

12. The method according to claim 8, wherein, the reaction step is switched to the regeneration step, based on variation of the catalyst temperature.

13. The method according to claim 8, wherein, the reaction step is switched to the regeneration step, based on yield of benzene produced in the reaction step.

14. The method according to claim 8, wherein the amount of addition of the carbon dioxide or carbon monoxide is 0.01% to 30% per volume of the lower hydrocarbon.
